(19) United States
(12) Patent Application Publication
(10) Pub. No.: US 2024/0238512 A1
(43) Pub. Date: Jul. 18, 2024

(54) VARIABLE DOSE THERAPEUTIC AGENT DISPENSER

(71) Applicant: NEUROCHASE TECHNOLOGIES LIMITED, Ocean Park, Cardiff (GB)

(72) Inventors: Steven Streatfield GILL, Bristol (GB); Thomas GILL, Bristol (GB)

(73) Assignee: NEUROCHASE TECHNOLOGIES LIMITED, Ocean Park, Cardiff (GB)

(21) Appl. No.: 18/288,676

(22) PCT Filed: Apr. 29, 2022
(86) PCT No.: PCT/GB2022/051102

§ 371 Date: Oct. 27, 2023

(30) Foreign Application Priority Data
Apr. 30, 2021 (GB) 2106203.9
Apr. 30, 2021 (GB) 2106210.4
Apr. 30, 2021 (GB) 2106224.5

(51) Int. Cl. A61M 5/145; A61M 5/14; A61M 5/142; A61M 5/162; A61M 5/168; A61M 5/19; A61M 5/38

(52) U.S. Cl. CPC A61M 5/14526; A61M 5/1413; A61M 5/14244; A61M 5/162; A61M 5/16881; A61M 5/19; A61M 5/385; A61M 2205/583; A61M 2207/00

(57) ABSTRACT

A variable volume fluid dispenser (10) for medical therapy and diagnostic use includes a tubular body (12) having a proximal end (16) and a distal end (14). Each end includes a fluidic connector (18, 26) and the tubular body (12) defines an interior volume. A piston (30) is located within the interior volume and is configured to create a fluid and gas seal within the interior volume of the tubular body (12) and to move axially under the action of a displacement fluid via the fluidic connectors (18, 26) between the proximal end (16) and the distal end (14). The position of the piston (30) relative to the distal end defines the volume of fluid to be dispensed.

### FIELD OF INVENTION

The present invention relates to a medical apparatus and in particular to an apparatus and an associated method for storing a predetermined volume of fluid, such as an infusate and/or therapeutic agent.

### BACKGROUND

Medical practice involves infusing precise volumes of fluid into the body for both diagnostic and therapeutic purposes. The routes of administration include, but are not limited to, intravenous, intra-arterial, intra peritoneal, intramuscular, intravitreal, intra tumoural, intra cystic, intrathecal, intra intra-cerebro-ventricular, and into the brain parenchyma via a technique called Convection Enhanced Delivery (CED).

Fluids containing agents infused for the diagnosis and monitoring of diseases that may be delivered via these routes include, but are not limited to, radio-opaque contrast agents for Magnetic Resonance Imaging (MRI), X-ray and X-ray Computerised Tomography (CT), radioisotopes for use in Single Photon Emission Computed Tomography (SPECT) and Positron Emission Tomography (PET) and dyes.

Fluids carrying therapeutic agents infused via these routes include, but are not limited to, chemotherapies, antibiotics, enzymes, neurotrophins, gene therapies, SiRNAs and antisense oligonucleotides, enzymes, immunomodulatory therapies (such as monoclonal antibodies and chimeric antigen receptor T-cell (CAR-T) therapy), Auger electron emitters, immunotoxins, molecular targeted therapies, monoclonal antibodies, oncolytic viruses, nanoparticles and botulinum toxin. Some of these therapies can be potentially hazardous, both to patients and to the clinical staff involved in preparing them for administration. Additionally, many of these therapies are very expensive.

There is a wide range of medical conditions potentially amenable to treatment by infusion of these agents via one or more of the above-referenced routes of administration. Examples of such medical conditions include, infectious diseases, malignant diseases; metabolic diseases, auto-immune and rheumatological diseases, enzyme deficiency diseases and neurological diseases.

Successful treatment of neurological diseases has been compromised by the limited access of therapies to the central nervous system when administered systemically. This is because of the presence of the blood-brain barrier (BBB). Nevertheless, even if one were able to freely cross the BBB with a systemically delivered therapy the ubiquitous effects of many potential therapies could cause significant toxicity. Successful treatment of many neurological diseases therefore requires the means to deliver effective therapeutic doses of drugs that are confined to the volume of pathological change in the central nervous system (CNS).

Direct delivery of therapies to clinically meaningful brain volumes can be accomplished using the CED method. Using the CED method, fluid containing therapeutic agents such as drugs, including viral vectors, proteins or chemotherapy are infused through judiciously placed micro cannulas at carefully controlled flow rates to carry the therapeutic agent homogeneously by bulk flow throughout the defined brain targets. The therapeutic agents/infusate is driven through the extracellular space in the tissue, replacing the extracellular fluid. The extracellular fluid constitutes approximately 20% of brain volume and so the volume of distribution per volume of infusion is approximately 5/1. Doses delivered by CED can be tightly controlled in the target volume where the BBB acts to retain the therapeutic agent/infusate giving long exposure times and resulting in negligible, if any systemic exposure.

Neurological diseases that may potentially be treated by direct infusion of therapeutic agent/infusate into the brain parenchyma using the method of CED include neurodegenerative diseases, enzyme deficient conditions, neuro-inflammatory diseases, neuro-oncological diseases and acquired neurological injuries. Depending on the specific pathology to be treated, infusions into several target volumes in an individual's brain may be required. For example, in the case of treating patients with Huntington's disease with a gene therapy, the patient's caudate nuclei and the putamen volumes would be targeted. Typically, one cannula would be delivered to each caudate and two cannulas would be required to fill each putamen. Thus, a total of six cannulas would be necessary. The volume of gene therapy required to treat patients with Huntington's disease will vary between individuals depending on the size of their respective nuclei, which in turn depends on their overall brain size and the degree of brain atrophy they have suffered. Hence the volume of infusion for each cannula to fill a given target in an individual needs to be calculated and prescribed.

Typical volumes infused into subcortical grey matter structures range between 200 and 800 μl per cannula but may be significantly more for general brain coverage. These larger volumes would be required for widespread brain diseases and brain tumours where 3-5 ml could be delivered per cannula.

As noted above, the therapies used for a wide range of medical conditions are often hazardous and/or expensive. The existing art for administering these medications to patients often involves the doctor, or another healthcare staff member, drawing the medication into a syringe, flushing the therapy through an infusion line before connecting it to an indwelling catheter or cannula in the patient. This is typically done on the ward, or in a related clinical space, such as in an operating theatre. In the case of therapies/infusate being delivered by CED and the distribution of infusate is monitored whilst the patient is in a MRI scanner the infusion line needs to be two to three metres long because the associated pump needs to be distant from the magnetic field. The syringe is loaded into a pump that is programmed to deliver the prescribed volume at an appropriate flow rate. Compliance in long infusion lines can result in unpredictable dosing because the initial flow of fluid in the line when the pump is turned on will cause expansion of the line i.e. increasing the line's internal volume. Furthermore, when the pump is switched off residual pressure in the line will cause bleed out from the line which may continue for some time. Following completion of the infusion the syringe and infusion line are disconnected from the cannula or catheter and disposed of in a hazardous waste container.

The risks associated with this procedure include, exposure of staff and/or patients, to hazardous medications or agents, wastage of expensive medications or agents in a syringe and long infusion line, and dosing errors. These risks are multiplied with complex dosing regimens as described above for delivering therapies into the brain through multiple cannulas simultaneously with each cannula possibly requiring a different volume of infusion.

WO 2013/117661 A2 provides an example of an apparatus for delivering therapeutic fluid to the brain. The apparatus includes cannulas inserted into the brain and a percutaneous fluid access device, which is anchored directly to the skull of the patient. The access device incorporates an external connector device which provides a fluidic link between external syringe pumps delivering therapies and the internal system incorporating the cannulas.

The external system includes a fluid storage apparatus that consists of a flexible plastic tube, which is sealed at both ends with a septum. The internal dimension of the tube (internal bore dimension and length) represents the internal volume and the volume of infusate i.e. the volume of therapeutic agent contained within the tube. Various tube lengths are considered such that various treatment volumes i.e. fixed volume sets can be available to allow selection of an appropriate tube when a specific treatment volume is required i.e. the selected tube is filled with the required dose of infusate. When delivery of the infusate is required, the appropriate tube can be connected proximally to a syringe (pre-primed with an inert fluid) via an infusion line with a hollow needle connector and distally a double ended hollow needle connector connects the fixed volume infusion line to a cannula, which includes a proximal septum seal.

The syringe is connected to an infusion pump that drives inert fluid through the fixed volume extension line thereby displacing the prescribed volume of the therapeutic agent into the cannula to be delivered into the brain tissue by convective flow and thence flushing the cannula's dead-space of residual therapy.

A shortcoming of this system is that the delivery of therapeutic agent to specific brain volumes is highly variable and, as described above, depends on the individual's brain size and more specifically to the size and shape of their individual brain nuclei. Particularly for gene therapies, highly accurate dosing may be required to prevent off target transfection and the development of long-term side effects. For example, volume differences of +/−20 μl may be desirable and so the necessary number of fixed volume sets (as described in WO 2013/117661 A2) to accommodate these volume differences make the described method impractical.

The fixed volume extension line described in WO 2013/117661 A2, when connected to an infusion line will result in mixing of the inert fluid and the therapeutic agent at the proximal end of the fixed volume extension.

### SUMMARY OF THE INVENTION

The present invention provides a variable volume fluid dispenser for medical therapy and diagnostic use, the variable volume fluid dispenser comprises:
a tubular body having a proximal end and a distal end, wherein the tubular body defines an interior volume between the proximal end and the distal end;
a fluidic connector on each of the proximal end and the distal end; and
a piston located within the interior volume, wherein the piston is configured to move axially between the proximal end and the distal end under the action of fluid displacement via the fluidic connectors, wherein the piston position relative to the distal end defines the volume of fluid to be dispensed.

In use the piston can divide the interior volume into a proximal volume and a distal volume when a volume of dispensing fluid less than the capacity of the interior volume is contained in the dispenser, as such the distal volume defines a dispensing chamber between the distal fluidic connector and a distal face of the piston. In use, the fluid contained in the dispensing chamber defines a predetermined volume of fluid, which can be stored within the variable volume dispenser until delivery/dispensing and wherein the volume of the dispensing chamber i.e. the distal volume depends on the position of the piston relative to the proximal end and the distal end.

The variable volume fluid dispenser addresses the problems associated with the safe dispensing, safe storage and accurate delivery of prescribed volumes of fluid, for the treatment, diagnosis and monitoring of diseases.

The variable volume aspect of the variable volume fluid dispenser is provided by movement of the piston to increase or reduce the distal volume, where the distal volume is defined between the distal end of the tubular body and a distal face of the piston. It will be appreciated the maximum volume of fluid contained in the variable volume fluid dispenser is when the piston abuts the proximal end. The proximal volume is defined between the proximal end of the tubular body and a proximal face of the piston.

The tubular body may be formed from a rigid pharmacologically inert material. For example, the tubular body may be made of glass or plastic. For example, the tubular body may be made of borosilicate glass (optionally type 1 borosilicate glass), such as Pyrex®. Alternatively, the tubular body may be made of laboratory grade polypropylene and/or polyethylene resins. The tubular body may be formed from a plastic such as cyclic olefin polymer (COP) or cyclic olefin copolymer (COC).

One or both fluidic connectors may comprise a hollow needle that makes a fluid connection by piercing a septum in a connecting member of apparatus to which the dispenser is being connected. Alternatively, one or both fluidic connectors may be a seal that is pierceable by a hollow needle. For example, the seal may be a septum stopper, which is pierceable by a hollow needle. Alternatively, one or both fluidic connectors may be a split septum that can be opened. For example, a device such as a blunt cannula or male Luer can penetrate the split septum to facilitate fluid flow via the split septum. Alternatively or additionally, one or both fluidic connectors may comprise a valve, for example which can be activated/opened by connection to an activating reciprocal connecting member. For example, the valve may be a mechanical valve, which requires physical activation of the valve to permit fluid flow. Alternatively or additionally, the valve may be a pressure sensitive valve, which is operable to open upon fluid flow (for example in response to a pressure gradient being established across the valve) and to close when fluid flow stops. The pressure sensitive valve may comprise a split seal or split septum. Alternatively or additionally, one or both fluidic connectors may comprise a needleless connector.

One or both fluidic connectors may further comprise a removable cap. In particular, where either of the fluidic connectors is a hollow needle the fluidic connector may further comprise a cap e.g. a removable transportation cap that hermetically seals the needle.

The proximal end fluidic connector may be configured to connect the proximal end of the dispenser to an infusion line, for example from a syringe pump. The infusion line may include a reciprocal connector to the proximal end fluidic connector. For example, the proximal end fluidic connector may include a septum and the reciprocal connector may include a hollow needle configured to pass through the septum.

The distal end fluidic connector may be configured to connect the distal end of the dispenser to a proximal end of a cannula, wherein the proximal end of the cannula includes a reciprocal connector to the distal end fluidic connector. For example, the distal end fluidic connector may include a hollow needle and the reciprocal connector may include a pierceable septum seal, wherein the hollow needle is configured to pierce the septum seal.

As mentioned, the piston can divide the interior volume into a proximal volume and a distal volume. The distal volume and the proximal volume may both be configured to hold relatively incompressible fluids, in particular liquids.

The proximal end connecting member and/or the distal end connecting member may be a Luer fitting, which is configured to connect with a reciprocal Luer fitting. Advantageously, a hollow needle connector housed within a male Luer fitting will protect the user from injury from the needle when handling the device.

In use, a double-ended hollow needle connector can be used for connection of the dispenser to a compatible member, for example a cannula or an infusion line, where the fluidic connector of the dispenser and the fluidic connectors of the compatible member/apparatus each include a pierceable seal.

The variable volume fluid dispenser according to the present invention provides an apparatus configured to contain, for delivery to a patient, a tailored volume of fluid for diagnostic or therapeutic purposes. The variable volume fluid dispenser may contain a therapeutic agent e.g. gene therapy to be delivered to a specific brain target volume.

As previously described, by way of example, in the treatment of Huntington's disease, six cannulas are required to be delivered to six targets i.e. two cannulas to each Putamen and one cannula to each caudate nucleus. It will be appreciated that nucleus size is variable between individuals and even between different sides of one individual's brain. As such, the volumes of therapeutic agent required to be delivered via each cannula is predetermined and therefore each dispenser associated with each cannula should be filled with a corresponding predetermined volume of fluid containing therapeutic agent to ensure safe and accurate delivery of the therapeutic agent to a patient. The variable volume dispenser fulfils this requirement because each dispenser can be configured to contain a predetermined volume of fluid containing therapeutic agent in the distal volume i.e. the dispensing chamber.

At least an axial portion of the tubular body may be transparent and include surface marking internally or externally to indicate the position of the distal end of the piston, thereby indicating the volume of fluid contained within tubular body. The marking may be a calibrated scale including graduated marks.

The piston may include a perimeter seal member operable to create a fluid and gas seal within the interior volume of the tubular body. The perimeter seal member may include one or more contact surfaces. For example, the perimeter seal may comprise an O-ring type seal, a lip seal, an elongated seal surface etc. The one or more contact surfaces of the perimeter seal may be configured with low friction properties, such that movement of the piston relative to the inner surface of the dispenser is free with little or no resistance when fluid acts on the distal or proximate surface of the piston. For example, at least an outer surface of the perimeter seal may include glass or polytetrafluoroethylene (PTFE). A PTFE or alternative fluoropolymer coating on the distal face of the piston would provide a drug compatible surface. The tubular body portion may also be silicone coated, or have a baked-in silicone coating, to reduce the fiction of the interacting surfaces.

Alternatively, the outer surface of the perimeter seal may include silicone.

A further aspect of the invention provides a method of filling the dispensing chamber of a variable volume fluid dispenser according to the first aspect, wherein the method comprises the steps of:
degassing the interior volume and priming the interior volume with an inert fluid via the proximal fluidic connector, wherein the piston is driven axially by the inert fluid until the interior volume is filled with inert fluid and the piston abuts the distal end;
attaching a receptacle of fluid to the distal end and an extractor to the proximal end;
extracting inert fluid from the interior volume whilst simultaneously drawing a predetermined volume of fluid into the distal volume from the receptacle, thereby filling the distal volume with a predetermined volume of fluid to be dispensed; and
disconnecting the extractor and the receptacle of fluid from the proximal end and distal end respectively.

The method may further comprise attaching a transportation cap over one or both of the distal end and the proximal end, thereby hermetically sealing the fluid contents of the variable volume fluid dispenser.

The step of extracting inert fluid from the interior volume whilst simultaneously drawing a predetermined volume of fluid into the distal volume can be carried out by creating a pressure gradient in either direction. Either the pressure can be reduced in the proximal volume by pumping out inert fluid via the proximal fluidic connector, or the pressure can be increased in the distal volume by pumping in fluid via the distal connector.

Inert fluid driven into or extracted from the proximal volume acts on the piston to decrease or increase the volume of fluid in the distal volume i.e. dispensing chamber, wherein the fluid in the dispensing chamber is the predetermined volume of fluid to be dispensed for therapeutic or diagnostic use.

### BRIEF DESCRIPTION OF THE DRAWINGS

An embodiment of the present invention will now be described with reference to the accompanying drawings in which:

FIG. 1 illustrates a variable volume therapeutic agent dispenser;

FIG. 2 illustrates a cross-section of the dispenser of FIG. 1 containing a predetermined volume of therapeutic agent and a volume of inert fluid;

FIG. 3 illustrates degassing and filling the dispenser of FIG. 1 with inert fluid;

FIG. 4 illustrates filling the dispenser of FIG. 1 with a fluid containing therapeutic agent;

FIG. 5 illustrates an exploded view of an application of the dispenser of FIG. 1;

FIG. 6 illustrates an exploded view of a septum sealed connector, provided at the proximal end of a cannula;

FIG. 7 illustrates a connecting member, which includes a Luer fitting, which is configured to connect with a reciprocal Luer fitting provided on the infusion line;

FIG. 8 shows an alternative design of septum sealed connector;

FIG. 9 shows an exploded view of the septum sealed connector of FIG. 8;

FIG. 10 shows a cross-sectional view of the septum sealed connector of FIGS. 8 and 9;

FIG. 11 shows a close-up of the septum sealed connector of FIG. 10 during use; and

FIG. 12 shows a wearable article for holding dispensers during use.

### DESCRIPTION

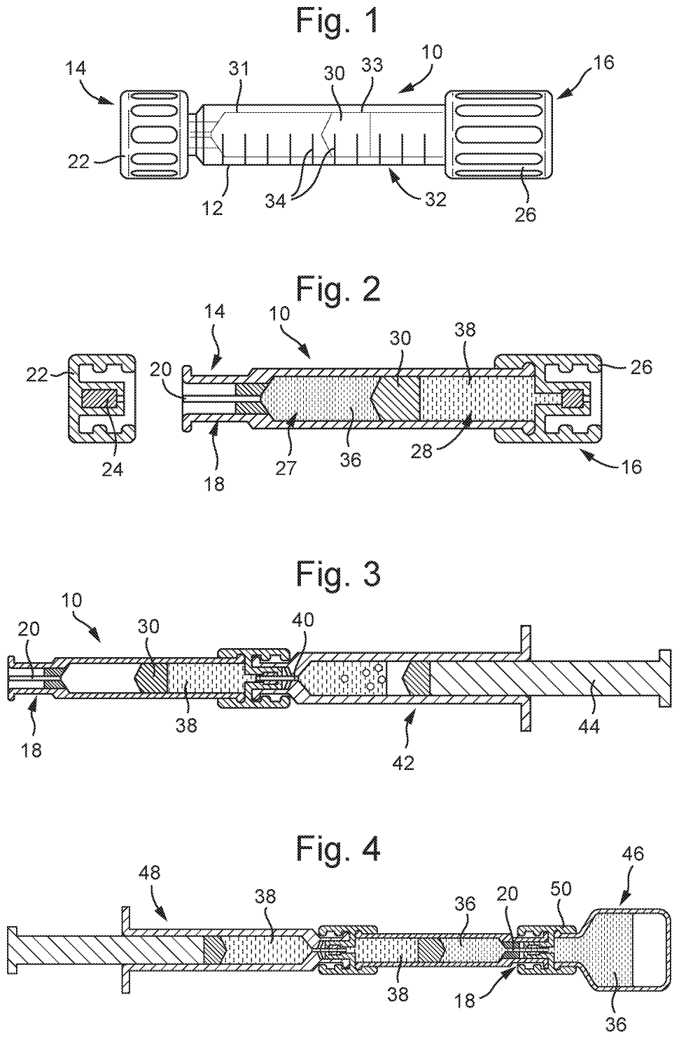

An example of a variable volume fluid dispenser 10 is illustrated in FIG. 1 and FIG. 2. The variable volume fluid dispenser 10 includes a tubular body 12, which is sealed at both ends. Hereafter, to distinguish one end of the tubular body 12 from the other, the ends will be referenced as a distal end 14 and a proximal end 16.

The distal end 14 and the proximal end 16 each include fluidic connectors 18, 26 which facilitate sealing the dispenser 10, when the tubular body contains inert fluid or dispensing fluid. One or both of the fluidic connectors 18, 26 can include a hollow needle connector, a seal, for example a septum seal that is pierceable by a hollow needle, a split septum that can be opened, for example by a blunt cannula or male Luer, which can penetrate the split septum to facilitate fluid flow. Alternatively, the fluidic connectors 18, 26 may include a valve, which can be activated/opened by connection to an activating reciprocal connecting member. The valve may be a mechanical valve, which requires physical activation of the valve to permit fluid flow. Alternatively, the valve may be a pressure sensitive valve, which is operable to open upon fluid flow and to close when fluid flow stops. Alternatively, one or both fluidic connectors may include a needleless connector. Examples of needless connectors are described in the article “Needleless Connectors: A Primer on Terminology” as published in the Journal of Infusion Nursing, January 2010—Volume 33—Issue 1—p 22-31. The fluidic connectors 18, 26 may be attached to the tubular body 12 by any suitable means, for example using an adhesive. The fluidic connectors 18, 26 may be attached to the tubular body using crimps. Preferably the crimps are made from a lightweight or low density material, for example aluminium, in order to minimise the overall weight of the dispenser 10.

In the illustrated example, the distal end 14 includes a fluidic connector 18, which incorporates a hollow needle 20 (see FIG. 2). To ensure the dispenser 10 and its contents are sealed, in the illustrated example, the distal end 14 also includes a transportation cap 22, which includes a silicon stopper 24. To hermetically seal the dispenser 10 the silicon stopper 24 is penetrated by the tip of the hollow needle 20 upon securing the cap 22 to the fluidic connector 18. The cap 22, when applied to the dispenser 10 prevents ingress from the atmosphere and ensures safe transportation of the dispenser 10.

In the illustrated example, the proximal end 16 incudes a fluidic connector 26. In this example, the fluidic connector 26 is a septum stopper, which provides a hermetic seal on the proximal end 16 thereby preventing ingress from the atmosphere and ensures safe and sterile transportation of the dispenser 10 when it contains fluid. It will be appreciated, the septum stopper 26, is configured for use in the usual manner, wherein it can be pierced by a hollow needle or cannulas to allow the contained fluid to be dispensed.

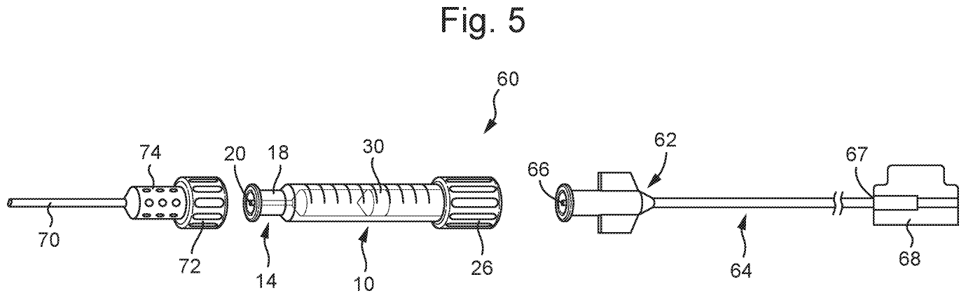

It will be appreciated that, instead of the illustrated hollow needle 20 and cap 22 arrangement as illustrated in FIG. 2 and described above, the distal end 14 may include a septum stopper. In this regard, it will be appreciated a double ended/tipped hollow needle (not illustrated) could be used to pierce the distal end septum stopper when used and another septum sealed connector (see FIG. 5) which is typically attached to a cannula, which facilitates delivery of the therapeutic fluid to the patient as the fluid is dispensed.

It will be appreciated, both the distal end 14 and proximal end 16 may include caps (not illustrated) to ensure the ends and content of the dispenser 10 remain sterile until required for delivery/dispensing the fluid contained in the dispenser 10.

The dispenser 10 includes an interior volume, which can be divided into a distal volume 27 and a proximal volume 28 by a moveable piston member 30. The moveable piston member 30 is a short cylinder, which is configured to move axially relative to the interior wall 31 of the tubular body 12. The moveable piston member 30 forms an interfacial fluid and gas seal between the outer sealing circumference/perimeter 33 of the piston 30 and the inner circumference/interior wall 31 of the tubular body 12.

The piston 30 is preferably made from a rigid material. This allows fluid dispensing and withdrawal to and from the dispenser 10 with minimal compliance of the piston 30, thereby improving the dosage precision and the responsiveness when dispensing. In the illustrated example, the piston 30 is made of glass (for example borosilicate glass), or a plastic material (for example Polytetrafluoroethylene, PTFE, or polyetheretherketone, PEEK) with an exterior perimeter seal 33 which provides surface contact thereby creating a fluid and gas seal within the interior volume of the dispenser body 12. At least the contact surface has low frictional resistance properties such that the piston 30 is displaced, unhindered, upon the action of fluid acting on an end face i.e. distal end 37 or proximate end 35 of the piston 30.

The configuration of the perimeter seal 33 may include one or more sealing contact surfaces. For example, the perimeter seal 33 may comprise one or more O-ring type seals, which are distributed along the width of the outer circumference of the piston 30 to ensure a fluid and gas seal within the interior volume of the piston 10. Other seals, for example lip seals formed on the circumference of the piston may be used as an alternative to O-ring seals. However, it will be appreciated the configuration of the piston circumference wall is such that a fluid and gas seal is created, and fluid ingress is prevented between the proximal volume 28 and the distal volume 27.

When the fluidic connectors 18, 26 are opened/activated to permit fluid flow to and from the dispenser 10, the piston 30 is configured to move in response to fluid being added to or dispensed from the dispenser 10. In this regard, the perimeter seal of the piston 30 is configured with low friction properties, such that movement of the piston 30 relative to the inner wall 31 of the dispenser 10 is free with little or no resistance when fluid acts on the distal surface 37 or proximate surface 35 of the piston 30. For example, at least an outer surface of the perimeter seal may include a material such as glass, polytetrafluoroethylene (PTFE), silicone or polyether ether ketone (PEEK).

Displacement of the piston 30 facilitates loading and dispensing fluid e.g. fluid containing therapeutic agent into and from the dispenser 10. Including the piston 30 within the interior volume permits storage of a predetermined maximum volume of fluid e.g. fluid containing therapeutic agent, but allows for variable volumes of fluid, up to the maximum volume of the dispenser 10 to be contained within the distal volume 27. The position of the moveable piston member 30, under the action of fluid displacement, relative to the distal end 14 defines the volume of fluid that can be contained and dispensed. The maximum interior volume of the dispenser 10 is defined between the distal end 14 of the dispenser 10 and the distal surface 37 of the piston 30, when the proximal end 35 of the piston 30 abuts the proximal end 16 of dispenser 10.

When the distal volume 27 contains a volume of fluid the distal volume 27 becomes the dispensing chamber, which is filled with a known volume of fluid e.g. fluid containing therapeutic agent. In the illustrated examples, see FIG. 2 and FIG. 4, the dispenser 10 contains a volume of inert fluid 38 and a volume of fluid containing therapeutic agent 36. The inert fluid 38 is contained in the proximal volume 28 and the fluid containing therapeutic agent 36 is contained in the distal volume 27. In this example the dispenser 10 includes a dispensing chamber (distal volume 27) and an inert chamber (proximal volume 28). The process of preparing the dispenser 10 is described below with reference to FIG. 3 and FIG. 4.

In the illustrated example, the tubular body 12 is transparent and is made of a rigid material, such as glass e.g. borosilicate glass (Pyrex®) or plastic e.g. laboratory grade polypropylene and/or polyethylene resins. The tubular body may be formed from a plastic such as cyclic olefin polymer (COP) or cyclic olefin copolymer (COC).

The body 12 includes a calibrated scale 32 including graduated marks 34 such that, visually, the position of the piston 30 and the amount of fluid containing therapeutic agent 36 contained in the dispensing chamber (distal volume 27) is easily identified. It will be appreciated that only an axial portion of the body 12 could be transparent and include the calibrated scale such that the position of piston 30 and the amount of therapeutic fluid is visible. Advantageously, the calibrated volume scale 32 on the dispenser 10 indicates accurately the true volume of fluid containing therapeutic agent 36 contained in the dispenser 10 to be infused making accurate volume delivery more certain. The distal end 37 of the piston 30 indicates on the scale 32 if there is fluid 36 remaining in the dispensing chamber i.e. distal volume 27. Therefore, when the fluid 36 is fully dispensed the distal end 37 of the piston 30 abuts the distal end 14 of the dispenser 10.

The variable volume dispenser 10 may be provided in a range of sizes that will accommodate predetermined maximum volumes, for example 0.5 ml, 1 ml, 2 ml, 3 ml, 4 ml and 5 ml. Each dispenser, as noted above and demonstrated below, may contain a volume of fluid that is less than the maximum capacity of the interior volume of the dispenser 10.

In the illustrated examples, FIG. 1 represents an empty dispenser 10 and FIG. 2 represents a dispenser 10 containing a predetermined volume of dispensing fluid i.e. therapeutic agent 36 and a volume of inert fluid 38. The inert fluid 38 may be a fluid that would have no biological effect or therapeutic effect if infused into the body, e.g. artificial cerebrospinal fluid (aCSF), normal saline or Hartman's solution. In most instances the inert fluid 38 acts purely as a hydraulic fluid and does not enter the body.

FIG. 3 illustrates the dispenser 10 being primed with aCSF. In this example, the interior volume of the dispenser 10 is degassed and primed with aCSF.

In the method illustrated in FIG. 3, the cap 22 is omitted from the distal end 14 and a hollow needle 40 of syringe 42 containing an inert fluid e.g. aCSF, pierces the septum stopper 26 of the proximal end 16. Depressing the plunger 44 of the syringe 42 displaces the inert fluid 38 from the syringe 42 into the dispenser 10 i.e. the inert fluid 38 fills the proximal volume 28 and displaces the piston 30 towards the distal end 14 of the dispenser 10. The action of the inert fluid 38 on the piston 30 also displaces gas from within the distal volume 27 via the hollow needle 20 at the distal end 14 of the dispenser 10. The process of filling the dispenser 10 with inert fluid displaces the piston 30 axially until the distal end 37 of the piston 30 abuts the distal end 14 of the dispenser 10 such that the interior volume of the dispenser is filled entirely with inert fluid 38.

After degassing and filling the dispenser 10 with an inert fluid 38 it is possible to load the dispensing chamber i.e. the distal volume 27 with a fluid containing therapeutic agent 36. This process is illustrated in FIG. 4.

FIG. 4 illustrates the creation of a therapeutic dispenser 10 illustrated in FIG. 2 i.e. a dispenser 10 in which the interior volume contains a predetermined/prescribed volume (distal volume 27) of fluid containing therapeutic agent 36 for dispensing and a volume (proximal volume 28) of inert fluid 38. In FIG. 4 a vial (container) 46 of fluid containing therapeutic agent 36 is attached to the distal end 14 of the dispenser 10 and an empty syringe (extractor) 48 is attached to the proximal end 16 of the dispenser 10.

In the illustrated example, the distal end 14 of the dispenser 10 includes a hollow needle 20, which pierces the illustrated septum stopper 50 on the vial (container) 46 of therapeutic fluid 36.

By withdrawing the plunger 44 of the syringe 42, the syringe 42 facilitates extracting inert fluid 38 from the interior volume of the dispenser 10 via the proximal end 16 whilst simultaneously drawing fluid containing therapeutic agent 36 into the dispenser 10 via the distal end 14. The action of extracting the inert fluid 38 acts upon the piston 30 to draw in fluid containing therapeutic agent 36 to create a dispensing chamber where a volume of fluid containing therapeutic agent 36 replaces an equal volume of inert fluid 38.

The step of extracting inert fluid from the interior volume whilst simultaneously drawing a predetermined volume of fluid into the distal volume can be carried out by creating a pressure gradient in either direction. Either the pressure can be reduced in the proximal volume by pumping out inert fluid via the proximal fluidic connector (as shown in FIG. 4), or the pressure can be increased in the distal volume by pumping in fluid via the distal connector.

The volume of fluid containing therapeutic agent 36 drawn into the dispenser 10 is indicated by the position of the distal end of the piston 30 aligning with the calibrated scale 32 provided on the wall of the tubular body 12.

When the dispenser 10 contains the required volume i.e. the prescribed volume of fluid containing therapeutic agent 36, the syringe 42 (extractor) and the vial (container) 46 can be disconnected from the dispenser 10. In the illustrated example, the distal end 14 of the dispenser 10 includes a hollow needle as the fluidic connector 18. As such, for safety and to ensure the contents of the dispenser 10 remains sterile and uncontaminated a transportation cap 22, which includes an internal seal/stopper 24 e.g. a silicon seal is fitted to the distal end 14. In the illustrated example, the tip of the hollow needle 20 pierces partway through the seal 24 provided as part of the transportation cap 22. In this configuration the dispenser 10 is hermetically sealed and can be transported safely.

From the above, it will be appreciated filling the dispenser 10 with a prescribed/predetermined dose of fluid containing therapeutic agent 36 can be carried out in advance of delivery by a trained pharmacist using an aseptic technique. It will be appreciated, fluid containing therapeutic agent, for example gene therapy requires preparation in a class Il biological safety cabinet.

FIG. 5 illustrates an example of an apparatus 60 required to dispense/deliver a predetermined volume of fluid 36 from the dispenser 10 using an aseptic technique.

In the illustrated example, the proximal end 16 of the dispenser 10 is connected to one end 62 of an infusion line 64, where the end 62 of the infusion line 64 includes a hollow needle connector 66, which pierces the septum stopper connector 26 on the proximal end 16 of the dispenser 10. A second end 67 of the infusion line 64 connects to a pump 68, e.g. a syringe pump.

Where the distal end 14 includes a transportation cap 22, the cap 22 is removed and the hollow needle 20 is exposed such that it can be connected to the means of delivering the fluid containing therapeutic agent 36 to a patient. In the illustrated example, the delivery means is a cannula 70, which extends from a septum sealed connector 72.

The cannula 70 may be an intracranial cannula which facilitates direct infusion of the fluid 36 into the brain.

After ensuring the cannula's septum seal connector 72 is clean/sterile, the distal end 14 of the dispenser 10 can be connected to the cannula 70 via the hollow needle connector 20. Once a fluid connection is established at both the proximal end 16 and the distal end 14 of the dispenser 10 the pump 68 can be activated.

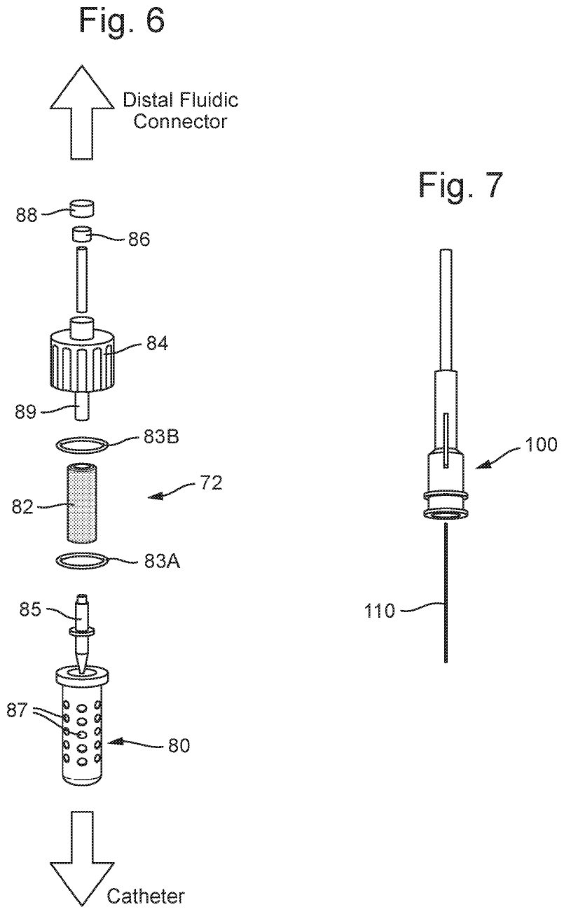

In the illustrated example, the septum sealed connector 72, which facilitates connection of the dispenser 10 and the cannula 70, includes a bubble vent 74. The configuration and component parts of the bubble vent 74 are described below and illustrated in FIG. 6. In summary, the bubble vent 74 mitigates the risk of bubbles entering the brain if they have come out of solution in the infusate or have been entrained in the infusate during the connection and/or disconnection of the variable volume dispenser 10 or an infusion line 64 to the cannula 70. It will be appreciated that bubbles infused into brain tissue can tear the tissue and disrupt distribution of the therapeutic fluid/infusate.

The septum sealed connector 72 is preferably provided at a proximal end of the cannula 70. The septum sealed connector 72 may be permanently joined to and/or integrally formed with the cannula 70, which further reduces the chance of bubbles being entrained during connection of the cannula 70 to the distal end fluidic connector 20. The septum sealed connector 72 may additionally be configured to prevent pathogens (for example micro-organisms such as bacteria) from entering the cannula 70. This reduces the risk of intracranial infections occurring due to the treatment.

FIG. 6 illustrates an exploded view of the septum sealed connector 72, configured for attachment to the cannula 70 and the distal end fluidic connector 20. The septum sealed connector 72 includes a perforated filter guard 80, a filter 82, retaining rings 83A, 83B, a retaining cap 84, a septum stopper 86 and a septum cap 88.

In the illustrated example, the filter 82 is a low volume bubble filter made from expanded polytetrafluoroethylene (ePTFE), which has a super-hydrophobic, gas permeable, microporous structure configured to remove bubbles from the flowing therapeutic fluid. In the illustrated example the filter 82 is effective to remove bubbles at flow rates of less than or equal to 30 μl/min. The filter 82 is contained in the perforated filter guard 80 and in the illustrated example the filter 82 is received over a hollow post 85 and is retained by a retainer ring 83A to the hollow post 85, which is positioned concentric to the filter guard 80.

In the illustrated example, the filter guard 80 is a hollow shell that includes a distribution of a plurality of perforations (small holes) 87 around the shell wall. The perforations 87 facilitate degassing the fluid as it flows through the filter 82 from the dispenser 10 to the cannula 70. The filter guard 80, as the name suggests also guards/protects the filter 82 against damage.

The combination of the filter 82 and the filter guard 80 facilitates dispersion of any entrained air/bubbles from the dispenser fluid flow before the fluid passes into the cannula 70.

The septum sealed connector 72 also includes a retaining cap 84, which connects with the filter guard 80 to complete the assembly of the sealed connector and to contain the filter 82 within the assembly. The retaining cap 84 includes a hollow retaining post 89 and a retainer ring 83B which engage with the filter 82 to ensure the filter 82 is correctly positioned and retained in the filter guard 80 to ensure efficient functionality of the filter 82 during use.

In the illustrated example, the retaining cap 84 includes a septum stopper 86, which provides a sealed unit until the septum 86 is pierced by a hollow needle thereby fluidly connecting the connector 72 to the cannula 70.

The septum stopper 86 is retained under compression by the septum cap 88.

In the illustrated example, the retaining cap 84 and filter guard 80 are joined by a snap fit connection. However, alternative arrangements could be used to join them together, for example a threaded connection, welded connection, glued connection etc.

The septum sealed connector 72 incorporating the low volume bubble filter 82, reduces the risk of air being delivered e.g. to the brain with the fluid containing therapeutic agent/infusate. It will be appreciated fluid containing air/bubbles will be space occupying and therefore is capable of stretching and tearing brain tissue whilst also disrupting delivery/distribution of the therapeutic agent/infusate. The septum sealed connector 72 can also act to filter out pathogens, including bacteria and other microorganisms from the fluid.

FIGS. 8 to 11 show an alternative design of septum sealed connector 174. FIG. 8 shows the septum sealed connector 174 in its assembled state ready for use. Similar to the septum sealed connector 72, the septum sealed connector 174 comprises a retaining cap 84. The retaining cap 84 comprises a proximal connector 176, for example a threaded connector, for connection to a fluid connector of the delivery system.

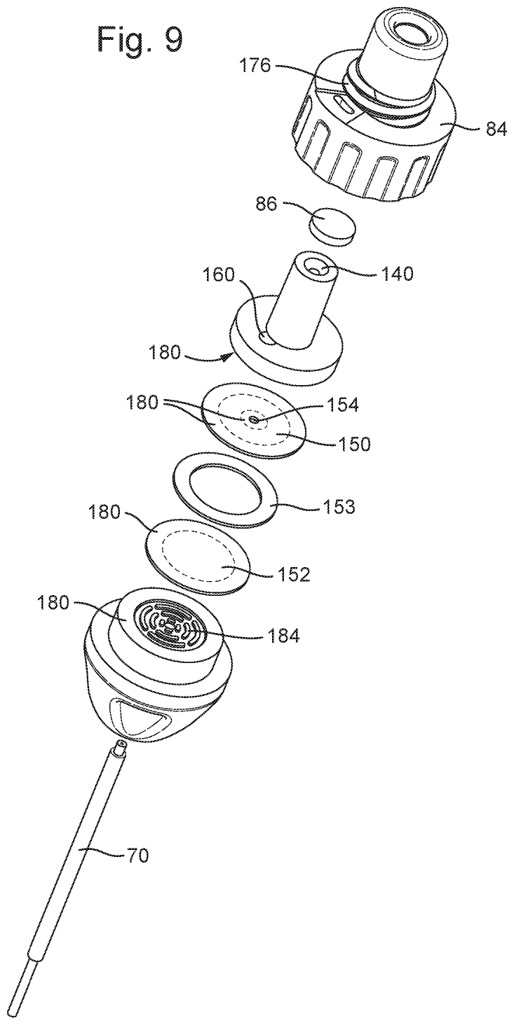

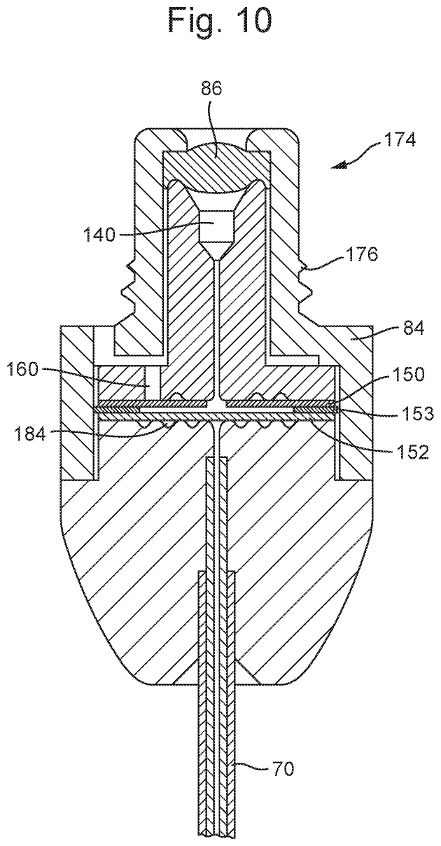

FIG. 9 shows an exploded view of the septum sealed connector 174, and FIG. 10 shows a cross-sectional view. The proximal connector 176 may comprise a septum 86 to seal the proximal connector 176 until the septum 86 is pierced, for example by a hollow needle. The septum sealed connector 174 comprises a fluid passage 140 fluidly connecting the proximal connector 176 and the cannula 70. In this design, the septum sealed connector 174 comprises a first membrane 150 and a second membrane 152. The first membrane 150 and the second membrane 152 are positioned between a distal end of the fluid passage 140 and a proximal end of the cannula 70. The first membrane 150 and the second membrane 152 may be substantially parallel to one another, and may be substantially perpendicular to an axis of the fluid passage 140. The first membrane 150 is positioned closer to the distal end of the fluid passage 140 than the second membrane 152, such that fluid entering the septum sealed connector 174 through the septum 86 reaches the first membrane 150 before the second membrane 152. An annular washer 153 may be positioned between the first membrane 150 and the second membrane 152 that forms a peripheral fluid seal between the membranes and the housing of the connector 174 and separates the membranes centrally to create a cylindrical gap between them. The cylindrical gap may have a diameter of between 2 mm and 6 mm but is most preferably 4 mm. The gap may separate the membranes 150 and 152 by 0.05 mm to 0.2 mm but most preferably by 0.1 mm. The first membrane 150 and the second membrane 152 may be connected to other components of the septum-sealed connector 174 via connection surfaces 180. The connection surfaces 180 may be joined by any suitable method, for example using ultrasonic welding or using an adhesive layer. The septum-sealed connector 174 may comprise a support member 184 to support the distal surface of the second membrane 152 and allow liquid that has passed through the second membrane 152 to more easily reach the cannula 70.

The first membrane 150 is hydrophobic and gas permeable. A hole 154 is provided in the first membrane 150 where the fluid passage 140 meets the first membrane 150, such that fluid from the fluid passage 140 can pass through the first membrane 150 via the hole 154. The septum sealed connector 174 may comprise a support member to support the proximal surface of the first membrane 150 and annular connection surfaces to attach the membrane around its periphery and around its central hole 154 (not shown in FIG. 9). The second membrane 152 is liquid permeable and preferably hydrophilic. It is not essential that the second membrane 152 is hydrophilic, however gas venting works most efficiently using the combination of a hydrophobic and a hydrophilic membrane. Use of the hydrophobic first membrane 150 alone could result in air being drawn from the atmosphere through the first membrane 150 and into the infusate if the pressure in the line falls below atmospheric pressure. This can happen if the connector is elevated above the head by more than 10-25 cm (depending on intracranial pressure). The second membrane 152 being hydrophilic prevents air ingress into the brain even in such situations. The second membrane 152 is impermeable to gas and bacteria. No hole is provided in the second membrane 152, such that fluid from the fluid passage 140 must pass through the material of the second membrane 152 to reach the cannula 70. At least one vent hole 160 (for example, two, three, four or more than four vent holes) is provided in the septum-sealed connector 174 on a proximal side of the first membrane 150. No holes are provided in the first membrane 150 where the vent holes 160 meet the first membrane 150, such that fluid from the fluid passage 140 must pass through the material of the first membrane 150 to reach the vent holes 160.

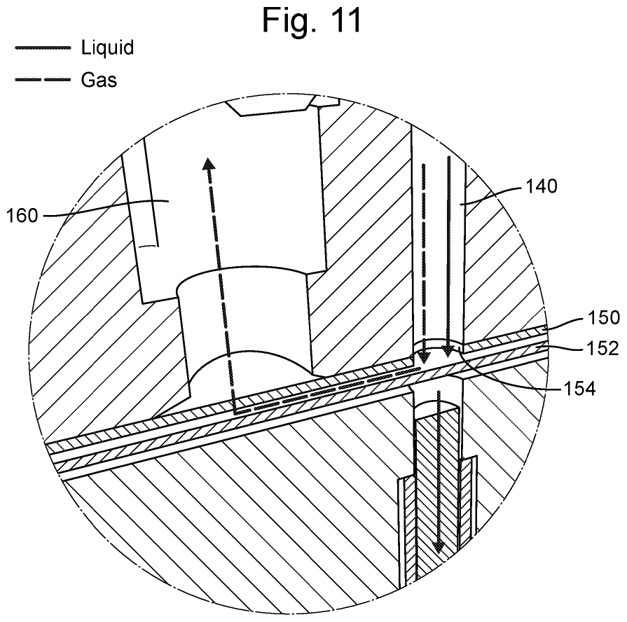

The operation of the septum-sealed connector 174 is demonstrated in the close-up view of FIG. 11. A mixture of liquid and gas (for example an infusate to be delivered to a patient's brain via the cannula 70 with some entrained bubbles of air) enters the septum-sealed connector 174 via the septum 86 and the fluid passage 140. The mixture passes through the first membrane 150 via the hole 154. The liquid is drawn to the hydrophilic second membrane 152, and soaks through the second membrane 152 (which is liquid permeable) into the cannula 70. The layer of liquid and the second membrane 152 form a barrier preventing gas from passing into the cannula 70. The gas will pass along the gap between the first membrane 150 and the second membrane 152, and can escape through the gas-permeable first membrane 150 at the position of one of the vents 160. The hydrophobic nature of the first membrane 150 repels liquid and prevents the liquid forming a similar barrier as on the second membrane 152, thereby allowing the gas to pass out of the septum-sealed connector 174 via the vent holes 160.

Advantageously, the tubular body 12 of the dispenser 10 is rigid, which means the configuration of the dispenser 10 is non-compliant/of low compliance to pressure build-up i.e. the tubular body 12 will not expand due to increased pressure. As such when the system 60, as illustrated in FIG. 5 has delivered the measured volume of fluid containing therapeutic agent 36, further movement of the piston 30 will be prevented by the distal end 14 of the dispenser 10. A feature of the rigidity of the tubular body 12 is that a sharp rise in pressure will be experienced within the interior volume of the dispenser 10 and the infusion line 64. The apparatus 60 will typically be configured such that any increase in pressure will raise a responsive alarm at the pump 68 as an indicator that the therapeutic agent 36 has been fully dispensed and to trigger an auto-stop feature in the pump 68.

It will be appreciated the alarm also indicates completion of the delivery of the therapeutic agent 36 to the therapist. Once the therapeutic agent 36 is fully dispensed, the therapist can disconnect the dispenser 10 from the catheter's septum sealed connector 72 and from the infusion line 64. Advantageously, when the infusion is complete the dispenser 10 is depleted of the therapeutic agent 36. Therefore, once disconnected the depleted dispenser 10 can be safely disposed of in an appropriate disposal receptacle e.g. a chemical/sharps bin.

To complete the process, fluid containing therapeutic agent may be flushed from dead space in the cannula 70. To do so the infusion line 64 can be connected directly to the cannula's septum seal connector 72 via the hollow needle connector 66A in the infusion line 64 and the pump 68 can be activated to flush the dead space therapeutic agent 36 with an inert fluid e.g. aCSF.

The above, with reference to FIG. 3, FIG. 4 and FIG. 5, demonstrates that the procedure to prepare and use the dispenser 10 is safe because of the straightforward, logical steps and minimal handling of the dispenser 10.

From the above, it will be appreciated due to the dispenser 10 containing a predetermined volume of therapeutic agent 36, overdosing by misprogramming an infusion pump 68 is avoided.

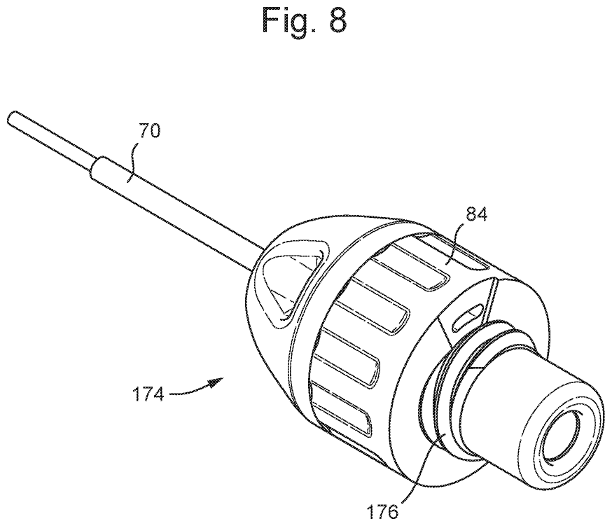

As described above, the proximal end fluidic connector includes a septum connector 26. FIG. 7 illustrates an example connecting member 100, which is a female Luer fitting, which is configured to connect the proximal end 16 of the dispenser 10 to an infusion line 64 from a syringe pump 68 (see FIG. 5). The Luer fitting 100 is a reciprocal connector containing a hollow needle 110 that passes through the septum 26. In the illustrated example, the Luer fitting 100 includes a hollow bullet tip needle 110, which is made from MRI compatible material, for example PEEK or titanium. The Luer fitting 100 retains the hollow needle 110 and can conceal the hollow needle 110 to reduce the risk of injury when handling the Luer fitting 100.

The distal end fluidic connector may include a Luer fitting, as illustrated in FIG. 7, where the Luer fitting 100 includes a hollow needle 110 which is configured to connect to a reciprocal Luer fitting, for example connector 72 (see FIG. 5), which includes a septum stopper on a proximal end of a catheter or cannula 70.

It will be appreciated from the above, in addition to the dispenser 10 being made of rigid/non-compliant material, the system would also benefit from an infusion line 64 made of non-compliant/low compliance material i.e. material resistant to expansion under pressure. For example, the infusion line may be an elongate tube made from material such as polyether ether ketone (PEEK). In an example, the outside diameter of the infusion line is 0.5 mm and an inner diameter in the range of 0.1 mm to 0.3 mm, but preferably in the range of 0.1 mm to 0.2 mm.

The combination of the rigid tubular body 12 (as described above) and non-compliant infusion lines reduces the risk of potential dosing inaccuracies.

The resulting hydraulic system, incorporating the dispenser 10 described above will thus be very sensitive to changes in pump pressure and flow rate. This is an important safety feature because in the event of a cannula 70 blockage, the resulting acute rise in pressure will trigger the pump alarm and the pump 68 will be switched off. In contrast, if the infusion line is compliant i.e. expands under pressure, the pressure rise will be gradual/slow, which will permit a significant volume of fluid to build up in the expanding infusion line. As such, if a cannula obstruction is overcome, e.g. a pellet of cored tissue is ejected, the resulting jet of fluid could cause local brain injury.

The example illustrated in FIG. 5 represents the dispenser 10 being used to deliver a fluid containing therapeutic agent 36 to a patient's brain. However, it should be appreciated that such a dispenser 10 could be used for the controlled infusions of discrete volumes of agents used for the diagnosis, monitoring and treatment of diseases in humans and animals. The device may be used for infusions of diagnostic and therapeutic agents into the body via one of many routes including intravenously, intra-arterially, intraperitoneally, intramuscularly, intravitreally, intracerebroventricularly, intrathecally, intraparenchymally, into tumours or cystic cavities.

Fluids containing agents infused for the diagnosis and monitoring of diseases that may be delivered via these routes using the variable volume dispenser 10 include but are not limited to, radio-opaque contrast agents for Magnetic Resonance Imaging (MRI), X ray and Xray Computerised Tomography, radioisotopes for use in Single Photon Emission Computed Tomography (SPECT) and Positron Emission Tomography (PET) and dyes.

Fluids containing therapeutic agents that may be delivered via these routes using the variable volume dispenser 10 include, but are not limited to, chemotherapies, antibiotics, enzymes, neurotrophins, gene therapies, SiRNAs and antisense oligonucleotides, enzymes, immunomodulatory therapies (such as monoclonal antibodies and chimeric antigen receptor T-cell (CAR-T) therapy), immunotoxins, botulinum toxin, molecular targeted therapies, monoclonal antibodies, oncolytic viruses, nanoparticles, therapeutic radionuclides including boron and auger electron emitters.

The variable volume therapeutic agent dispenser 10 exemplified in FIG. 1 to FIG. 5 and described above has many safety features, which are advantageous over existing methods of delivering potentially hazardous diagnostic and therapeutic agents/drugs such as gene therapies, chemotherapies and radioisotopes. For example, a dispenser 10 can be filled with the prescribed volume of a therapeutic or diagnostic agent 36 in the controlled environment of a pharmacy by a trained pharmacist, where necessary in a biological and/or radiation safe cabinet. As noted, after the dispenser 10 is filled it can then be hermetically sealed with the application of a transport cap 22 applied to the hollow needle connector 18 on its distal end. The dispenser can now be labelled and be safely transported to the location e.g. a hospital ward or MRI suite where the fluid containing the diagnostic or therapeutic agent 36 can be delivered. Upon receiving and checking the labelled contents of the dispenser against the patient's details and prescription the fluidic connector on the patient's cannula or catheter is cleaned, the cap 22 is removed and the hollow needle connector 18 on the distal end 14 of the dispenser 10 is connected to the cannula or catheter's septum sealed connector. An infusion line primed with inert fluid and connected to an infusion pump is connected to the fluidic connector 26 on the proximal end of the dispenser via its distal connector 62 and the pump switched on.

There are evident safety advantages to this described method over a doctor drawing up a biohazardous material from a vial into a syringe on a hospital ward, removing bubbles from the syringe then priming an infusion line and connecting it to a patient's catheter or cannula, loading the syringe in a pump and programming the pump to deliver the prescribed volume at an appropriate flow rate. The risks include human error in dispensing the prescription in a stressed clinical environment such as a hospital ward, needle stick injury, contamination of the clinical environment from spillage when dispensing and exposure of the biohazardous material to staff and patients. In the circumstances where the prescribed therapy is dispensed by a pharmacist into a syringe which is then transported to a ward, accidental spillage can occur when priming and de-gassing infusion lines or there is inadvertent depression of the syringe plunger when being loaded into a syringe pump.

Use of the variable volume dispenser 10 has particular advantage in minimising the dead-space in the infusion system. The device is typically connected directly to the cannula or catheter so that there is a relatively short distance to the target and the diagnostic or therapeutic agent is separated from the infusion pump and potentially long infusion line by the piston. This minimises the loss of potentially valuable therapy in the dead-space of an infusion line and syringe and minimises loss of therapy which may become bound to the material of an infusion line. For viral vectors this loss may amount to greater than 10% of the administered dose. The use of a rigid e.g. glass variable volume dispenser which has very low viral vector binding in combination with a relatively short cannula will minimise this loss. The variable volume dispenser will be provided in a range of sizes such as 0.5 ml, 1.0 ml, 2.0 ml, 3 ml, 4 ml, and 5 ml, for example, so that the size of the device can be tailored to the therapy being delivered, further minimising wastage. This is in contrast to clinically available infusion pumps which typically accommodate a limited range and type of compliant plastic rather than glass syringes such as 2 ml, 5 ml, 10 ml, 20 ml and 50 ml. Nevertheless, therapy may be delivered by the glass variable volume dispenser 10 when connected to a standard infusion pump driving a standard plastic syringe. By way of example a typical volume of a gene therapy to be delivered into a brain target from a CED cannula would be 300 μl. This could be loaded into the distal, dispensing end of a 0.5 ml, rigid/non-compliant variable volume dispenser 10 and connected to a CED cannula. The proximal end of the dispenser 10 may then be connected to a 2 ml plastic syringe and extension line filled with inert fluid and a standard medical syringe pump then used to deliver the therapy (see FIG. 5).

As described above, the dispenser 10 is rigid and is made from glass or plastic, which makes the dispenser 10 MRI compatible. As such the dispenser 10 can be safely and reliably placed within an MRI coil, such as a head coil. In addition, where possible, using low compliance materials for the infusion lines 64, means the infusion lines can be as long as required to ensure the infusion pump 68 is located a safe distance away from the magnetic field. Using low compliant infusion lines between the syringe in the infusion pump and the variable volume dispenser 10, for example using PEEK tubing with an internal diameter of 0.1 mm to 0.2 mm has the advantage of creating a highly responsive hydraulic system to drive the delivery of the therapy. This increases the accuracy of delivery as variable volumes of an infused therapy can be lost in the dead space of a compliant line that stretches when pressurised. In addition, the rapid rise in pressure in a noncompliant line resulting from an obstructed cannula, would cause the pump to alarm and switch off thereby making the system safer.

The dispenser 10 is particularly suited to complex dosing regimens, for example for delivering different volumes of fluid containing therapeutic agents into a patient's brain through multiple cannulas simultaneously. For some treatments this may require delivery through six or eight cannulas. In this instance a corresponding number of variable volume dispensers 10 containing prescribed volumes of therapy can each be connected to their assigned cannula and each can be connected to a separate infusion line and syringe pump. If all the pumps are set to a predetermined flow rate then the clinician overseeing the treatment can turn them on at random and when the prescribed volume in any dispenser 10 has been delivered the piston 30 will be stopped when it engages with the distal end 14 of the dispenser 10 causing a pressure rise in the proximal chamber 28 and infusion line causing the pump to alarm and switch off. When all pumps have alarmed and switched off the therapy will have been delivered. This can be confirmed by the clinician inspecting the position of the piston 30 relative to the marked scale 32 on each dispenser 10. When depleted of fluid, each dispenser 10 can be disconnected from their respective cannulas and disposed of in an appropriate clinical waste container. This process greatly simplifies the method of delivery, making it safer and reducing the likelihood of human error.

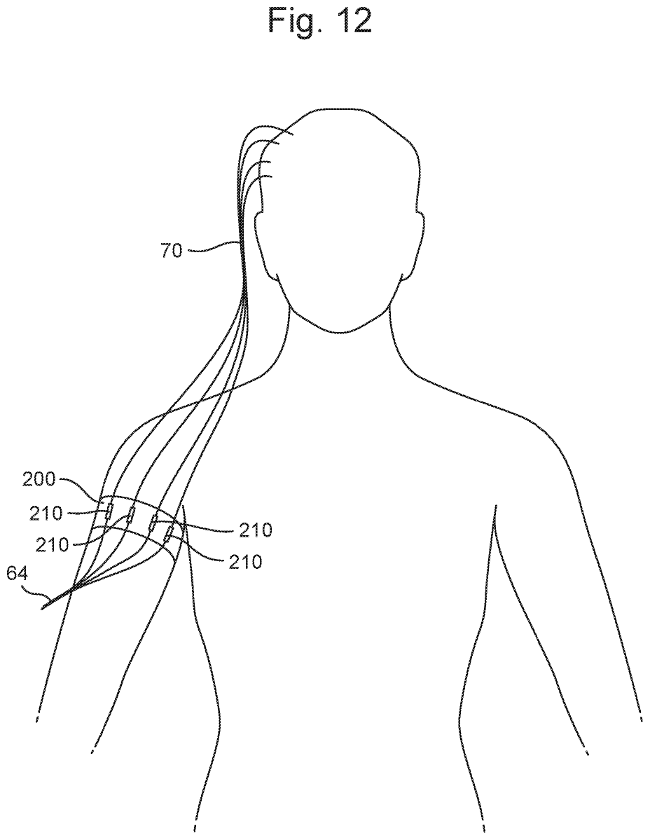

Dispensing in some procedures may take hours, and so it is preferable that the dispenser 10 is held securely and conveniently during the procedure. This is particularly true when plural dispensers 10 are used simultaneously. Holding the dispensers 10 in this way reduces the likelihood of damage to the dispensers 10 and improves comfort for the patient. For this purpose, there may be provided a wearable article 200 configured to hold one or more dispensers 10. FIG. 12 shows an example of a wearable article 200. Preferably, the wearable article 200 can hold a plurality of dispensers 10, for example two, four, six, eight, or more than eight dispensers 10. The wearable article 200 may be configured to be worn around the head, arm, or chest of a patient. The wearable article 200 may be provided in the form of a small item of clothing that can be worn in addition to a patient's normal clothing, for example a headband, a belt, or a sash. For example, the wearable article 200 shown in FIG. 12 is in the form of a band worn around the patient's arm. The wearable article 200 may alternatively be provided as a larger article of clothing, for example a jacket, coat, shirt, or hat.

The wearable article 200 may be adjustable so that it can be fitted securely to a patient regardless of their size. The wearable article 200 is preferably configured to hold the dispensers 10 in a position where they are less likely to interfere with a patient's movement and/or interfere with any diagnostic procedures that may be carried out on the patient during a dispensing procedure, such as x-ray or magnetic resonance imaging scans. For example, the wearable article 200 may be configured to hold the dispensers on the patient's upper chest or upper arm, or on the patient's forehead. The wearable article 200 may also comprise one or more straps or closures to hold the infusion lines 64 and/or cannulas 70 that connect to the dispensers 10.

The wearable article 200 may comprise one or more dispenser holders 210, each dispenser holder 210 configured to hold one dispenser 10. The dispenser holders 210 may comprise a compartment or retaining loop in which the dispenser 10 can be placed. The dispenser holders 210 may be adjustable, to allow the dispensers 10 to be easily inserted and securely retained. The wearable article 200 and/or the dispenser holders 210 may be adjustable by the use of any suitable means, for example one or more of elastic, an adjustable fastening such as a belt, buckle, or strap, or a hook and loop fastener. Alternatively or additionally, the dispenser holders 210 may be configured to be opened and closed, for example using a flap or lid. Where the dispenser holders 210 comprise a compartment, the dispenser holders 210 are configured such that the infusion line 64 and cannula 70 can pass out of the compartment even when the compartment is closed.

The dispenser holders 210 may be individually labelled with a unique identifier, for example a number, letter, or colour, that distinguishes each dispenser holder 210 from the other dispenser holders 210. This allows the dispensers 10 to be easily and quickly identified during a dispensing procedure, for example if a dispenser 10 becomes empty and needs to be replaced with a new dispenser 10 filled with the same therapeutic agent.

When multiple cannulas are implanted and connected to dispensers 10, management of the tubing between the head and the dispenser holders 210 may be assisted by the provision of a cable tie, strap, or a spiral wrap.

In summary many diagnostic agents and therapies requiring infusion into the body for a wide range of medical conditions are often hazardous and/or expensive. Therefore, using the dispenser 10, method and system 60, as described above with reference to FIGS. 1 to 7 reduces exposure of staff, and/or patients, to hazardous medications or agents, reduces wastage of expensive medications or agents in a syringe and long infusion line, reduces the risk of dosing errors because the dispenser 10 is configured to contain accurate doses and removes any guess work because when the dispenser 10 is depleted of fluid there is certainty that an accurate dose of therapy has been dispensed.

Whilst specific embodiments of the present invention have been described above, it will be appreciated that departures from the described embodiments may still fall within the scope of the claims.

1. A variable volume fluid dispenser for medical therapy and diagnostic use, the variable volume fluid dispenser comprises:
a tubular body having a proximal end and a distal end, wherein the tubular body defines an interior volume between the proximal end and the distal end;
a fluidic connector on each of the proximal end and the distal end; and
a piston located within the interior volume, wherein the piston is configured to move axially between the proximal end and the distal end under the action of fluid displacement via the fluidic connectors, wherein the piston position relative to the distal end defines the volume of fluid to be dispensed.

2. The dispenser as claimed in claim 1, wherein the piston is operable to divide the interior volume into a proximal volume and a distal volume, wherein a volume of dispensing fluid, which is less than the capacity of the interior volume is contained in the distal volume.

3. The dispenser as claimed in claim 1, wherein the tubular body is formed from a rigid pharmacologically inert material.

4. The dispenser as claimed in claim 1, wherein the tubular body is made of glass.

5. The dispenser as claimed in claim 4, wherein the tubular body is made of borosilicate glass.

6. The dispenser as claimed in claim 1, wherein the tubular body is made of plastic.

7. The dispenser as claimed in claim 6, wherein the tubular body is made of laboratory grade polypropylene and/or polyethylene resins.

8. The dispenser as claimed in claim 1, wherein one or both fluidic connectors comprise a hollow needle.

9. The dispenser as claimed in claim 1, wherein one or both fluidic connectors comprise a seal pierceable by a hollow needle.

10. The dispenser as claimed in claim 1, wherein one or both fluidic connectors comprise a split seal that can be opened to facilitate fluid flow.

11. The dispenser as claimed in claim 1, wherein one or both fluidic connectors comprise a valve, which can be opened/activated by connection to an activating reciprocal connecting member.

12. The dispenser as claimed in claim 11, wherein the valve is a mechanical valve, which requires physical activation of the valve to permit fluid flow.

13. The dispenser as claimed in claim 11, wherein the valve is a pressure sensitive valve, which is operable to open upon fluid flow and to close when fluid flow stops.

14. The dispenser as claimed in claim 1, wherein one or both fluidic connectors comprise a needleless connector.

15. The dispenser as claimed in claim 1, wherein one or both fluidic connectors further comprise a removable cap.

16. The dispenser as claimed in claim 1, wherein the proximal end fluidic connector comprises a septum stopper pierceable by a hollow needle.

17. The dispenser as claimed in claim 1, wherein the distal end fluidic connector includes a connecting member incorporating a hollow needle configured to pierce a pierceable reciprocal connecting member provided on apparatus to which the dispenser is to be connected.

18. The dispenser as claimed in claim 1, wherein the proximal end connecting member is a Luer fitting, which is configured to connect with a reciprocal Luer fitting.

19. The dispenser as claimed in claim 1, wherein the distal end connecting member is a Luer fitting, which is configured to connect with a reciprocal Luer fitting.

20. The dispenser as claimed in claim 1, wherein at least an axial portion of the tubular body is transparent and includes markings internally or externally to indicate the position of the distal end of the piston, thereby indicating the volume of fluid contained within tubular body.

21. The dispenser as claimed in claim 20, wherein the markings include a calibrated scale including graduated marks.

22. The dispenser as claimed in claim 1, wherein the piston includes a perimeter seal member operable to create a fluid and gas seal within the interior volume of the tubular body.

23. The dispenser as claimed in claim 22, wherein the perimeter seal member includes one or more contact surfaces.

24. The dispenser as claimed in claim 22, wherein the perimeter seal is configured with low friction properties, such that movement of the piston relative to the inner surface of the dispenser is free with very little resistance when fluid acts on the distal or proximate surface of the piston.

25. The dispenser as claimed in claim 24, wherein at least an outer surface of the perimeter seal includes glass.

26. The dispenser as claimed in claim 24, wherein at least an outer surface of the perimeter seal includes polytetrafluoroethylene (PTFE).

27. The dispenser as claimed in claim 24, wherein at least an outer surface of the perimeter seal includes silicone.

28. The dispenser as claimed in claim 24, wherein at least an outer surface of the perimeter seal includes polyether ether ketone (PEEK).

29. The dispenser of claim 1, further comprising a cannula for connection to the distal end fluidic connector.

30. The neurosurgical apparatus of claim 29, wherein the cannula comprises a septum-sealed connector configured to prevent gas and/or micro-organisms from entering the cannula.

31. The neurosurgical apparatus of claim 30, wherein the septum-sealed connector comprises a first membrane and a second membrane separated by an air gap, wherein the first membrane is hydrophobic and the second membrane is hydrophilic.

32. A method of filling the dispensing chamber of a variable volume fluid dispenser comprising a tubular body having a proximal end and a distal end, wherein the tubular body defines an interior volume between the proximal end and the distal end;
a fluidic connector on each of the proximal end and the distal end; and
a piston located within the interior volume, wherein the piston is configured to move axially between the proximal end and the distal end under the action of fluid displacement via the fluidic connectors, wherein the piston position relative to the distal end defines the volume of fluid to be dispensed.
wherein the method comprises the steps of:
degassing the interior volume and priming the interior volume with an inert fluid via the proximal fluidic connector, wherein the piston is driven axially by the inert fluid until the interior volume is filled with inert fluid and the piston abuts the distal end;
attaching a receptacle of fluid to the distal end and an extractor to the proximal end;
extracting inert fluid from the interior volume whilst simultaneously drawing a predetermined volume of fluid into the distal volume from the receptacle, thereby filling the distal volume with a predetermined volume of fluid to be dispensed; and
disconnecting the extractor and the receptacle of fluid from the proximal end and distal end respectively.

33. The method as claimed in claim 32, further comprising attaching a transportation cap over one or both of the distal end and the proximal end, thereby hermetically sealing the fluid contents of the variable volume fluid dispenser.
